(19) United States
(12) Patent Application Publication
(10) Pub. No.: US 2011/0015578 A1
(43) Pub. Date: Jan. 20, 2011

(54) TAMPER-PROOF CLOSURE HAVING A RETAINING PIN

(75) Inventor: Christiane Lowke, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Bayer Schering Pharma Aktiengesellschaft, Berlin (DE)

(21) Appl. No.: 12/921,208

(22) PCT Filed: Feb. 24, 2009
(86) PCT No.: PCT/EP2009/001290

§ 371 Date: Sep. 7, 2010

(30) Foreign Application Priority Data
Mar. 7, 2008 (DE) 10 2008 013 198.9

(51) Int. Cl. A61M 5/32; A61J 1/14

(52) U.S. Cl. 604/192; 604/403

(57) ABSTRACT

The invention relates to a tamperproof seal for a container or a syringe, with a lockable conical adapter which is sealed in a sterile manner by means of a rubber stopper and is protected by a safety sleeve, wherein the safety sleeve, which is pushed non-releasably onto the conical adapter lock, comprises a retaining part, a break-off part and a break-off zone located between said parts. The break-off part encloses the rubber stopper without radical contact. It has a retaining pin, the free end of which rests axially on the rubber stopper—when the tamperproof seal is fitted, and it has at least two open apertures. The length of the break-off part corresponds to at least twice the length of the internal thread of the conical adapter lock.

The present invention produces a tamperproof seal which ensures safe handling of the combination comprising the container or syringe and tamperproof seal.

The invention relates to a tamperproof seal for a container or a syringe, with a lockable conical adapter which is sealed in a sterile manner by means of a rubber stopper and is protected by a safety sleeve, wherein the safety sleeve, which is pushed non-releasably onto the conical adapter lock, comprises a retaining part, a break-off part and a break-off zone located between said parts.

U.S. Pat. No. 6,520, 935 B1 discloses a tamperproof seal of this type. In the subject matter therein, the rubber stopper sits in the break-off part in a form-fitting manner. As a result, the filled container bearing the tamperproof seal is completely opened upon the break-off part being broken off or torn off.

EP 1 034 810 B1 likewise discloses a tamperproof seal with a safety sleeve which can be partially removed by being broken off. In this case too, the rubber stopper is pulled from the conical adapter when the break-off part is broken off.

The present invention is based on the problem of producing a tamperproof seal for a container or a syringe, which tamperproof seal ensures safe handling of the combination comprising the container or syringe and tamperproof seal and which can be partially removed in a simple manner such that the container or the syringe is still sealed in a sterile manner thereafter.

The problem is solved by the features of Claim 1. To this end, the break-off part of the safety cap surrounds the rubber stopper without radial contact. The break-off part has a retaining pin, the free end of which rests axially on the rubber stopper—when the tamperproof seal is fitted. The break-off part has at least two open apertures. The length of the break-off part corresponds to at least twice the length of the internal thread of the conical adapter lock.

The present tamperproof seal is used for filled containers or syringes. In the present case, it guarantees, for example, safe and sterile sealing of a cartridge filled with a diagnostic means for magnetic resonance tomography. Like many other prefilled containers, this cartridge, including its tamperproof seal, also has to be brought into a sterile condition by autoclave treatment. In order to ensure the sterility of the assembly comprising the container and tamperproof seal, liquid residues must not remain behind in the region of the tamperproof seal during the drying operation. In order to prevent this, the parts involved: the internal thread adapter, the rubber stopper and safety cap are in contact predominantly via linear and/or spot-type contact. Large capillary gaps in which residual moisture from the autoclave treatment could be troublesomely retained are prevented.

The safety cap of the tamperproof seal has a break-off part which surrounds the rear part of the rubber stopper without coming into contact therewith in the radial direction, with respect to the centre line of the tamperproof seal. However, said safety cap presses the rubber stopper in a regaining manner against the outer cone of the lockable conical adapter. By means of this configuration, it is possible to separate or start to tear off the break-off part of the safety cap from the retaining part thereof without the rubber stopper being pulled off the outer cone.

Further details of the invention emerge from the dependent claims and the description below of a schematically illustrated embodiment.

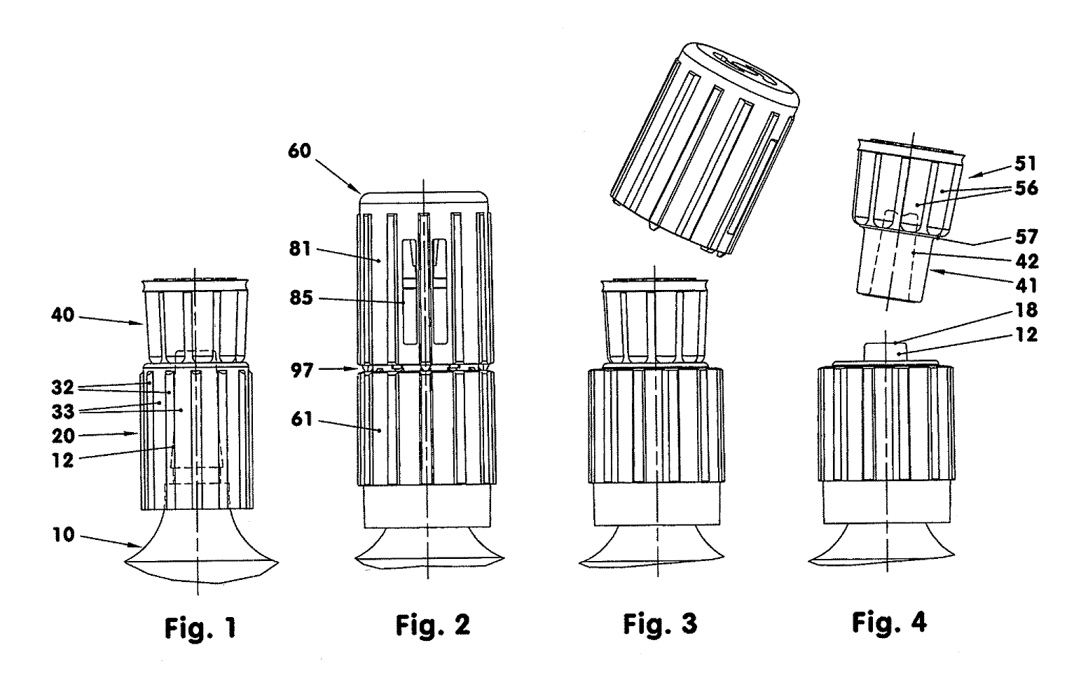

FIG. 1: a syringe with an internal thread adapter and rubber stopper placed thereon;

FIG. 2: a safety cap, placed over the parts from FIG. 1;

FIG. 3: the upper part is broken off from the safety cap to release it;

FIG. 4: open syringe, the rubber stopper has been removed;

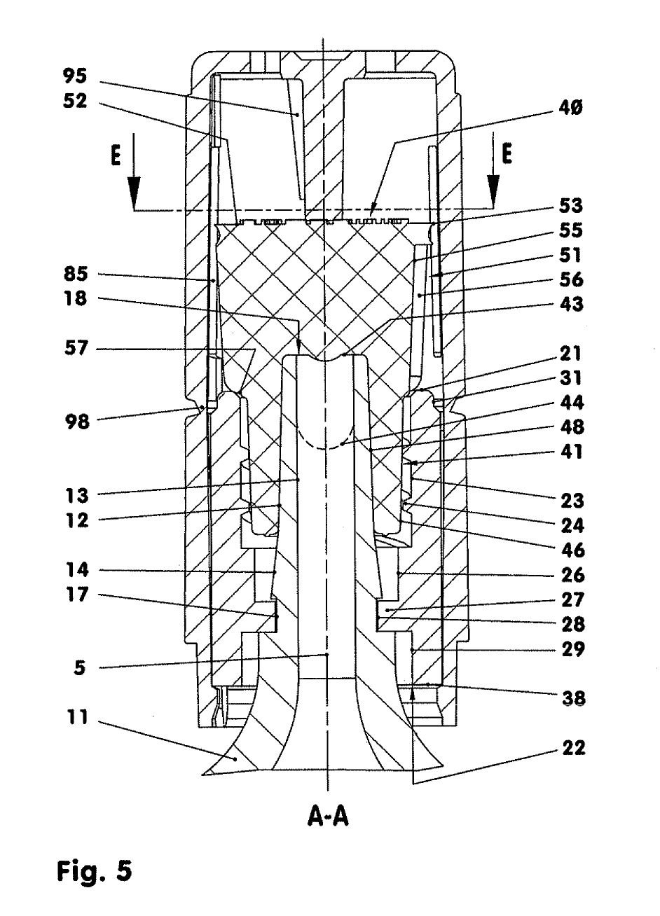

FIG. 5: longitudinal section A-A, transversely to the illustration according to FIG. 2;

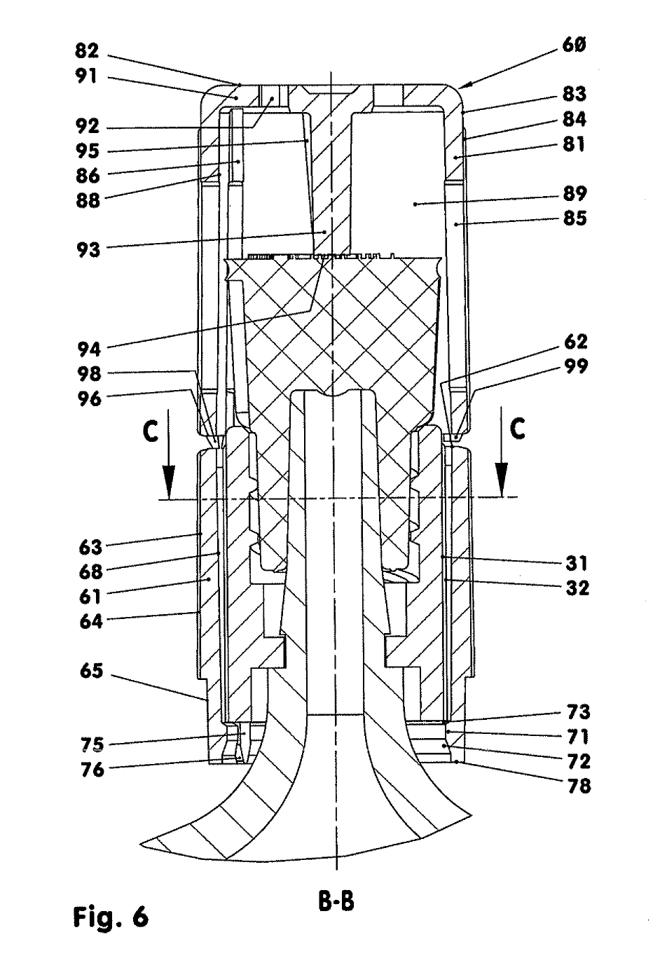

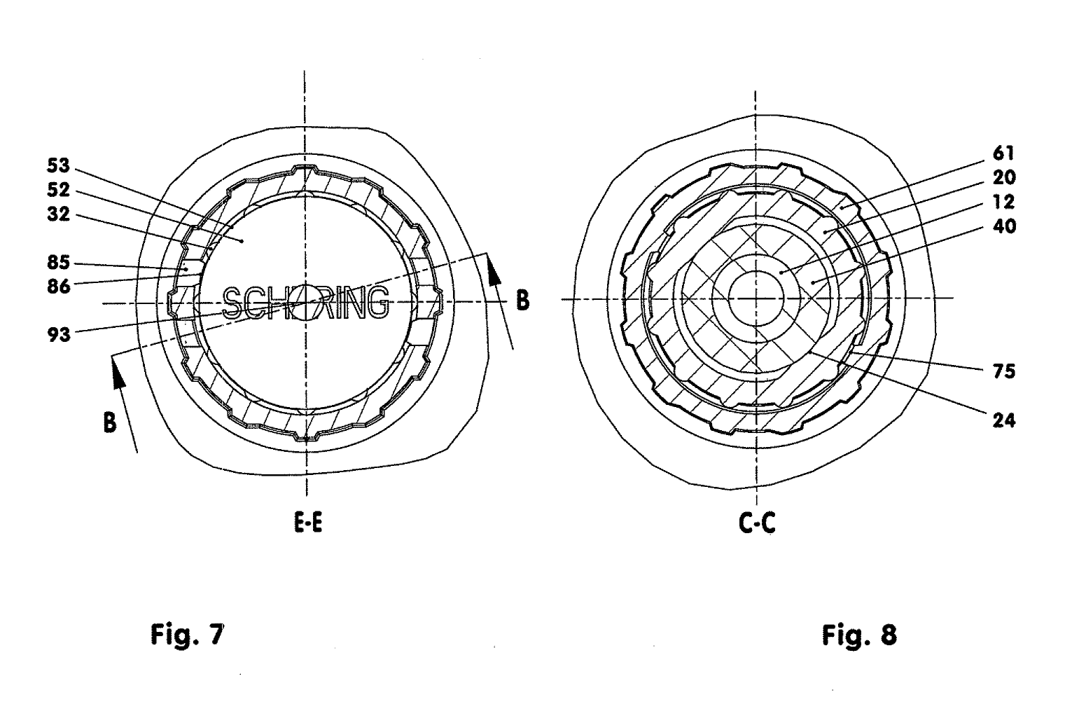

FIG. 6: longitudinal section B-B, cf. the sectional profile according to FIG. 7;

FIG. 7: cross section through the safety cap, cf. the sectional profile according to FIG. 5;

FIG. 8: cross section through the safety cap, the lockable conical adapter and the rubber stopper, cf. the sectional profile according to FIG. 6;

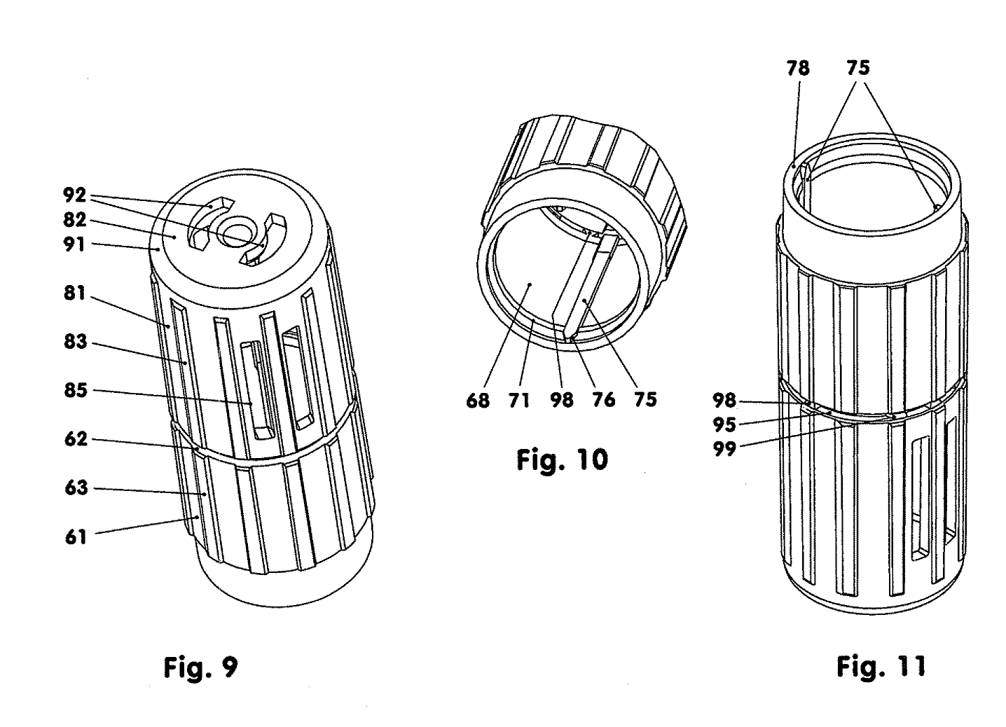

FIG. 9: perspective illustration of the safety cap obliquely from above;

FIG. 10: interior view of the safety cap;

FIG. 11: as FIG. 9, but pivoted through an angle of approx. 170 degrees.

In the exemplary embodiment, the present tamperproof seal serves to seal a syringe barrel (10) in a sterile manner. To this end, the syringe barrel (10) has a lockable conical connection in the form of an outer cone (12) with an internal thread adapter (20) which can be latched into position. The outer cone (12) is sealed by a rubber stopper (40), cf. FIG. 1. A safety cap (60) is pulled over the internal thread adapter (20) and the rubber stopper (40), cf. FIG. 2.

According to FIG. 5, the syringe barrel (10) has a barrel base (11) which merges in the form of a funnel into an outer cone (12). The outer cone (12), for example a Luer outer cone according to DIN 13090, Part 1, has a central discharge bore (13). The discharge bore (13) has a cross section of, for example, 5.6 mm².

A truncated cone section (14) which has a cone angle of approx. 14 degrees and tapers in the direction of the outer cone (12) is located below the outer cone (12). The length of the truncated cone section (14) is approx. ⅓ of the length of the Luer outer cone.

An encircling annular channel (17), the channel base of which is in the shape of a barrel casing, is located between the barrel base (11) and the truncated cone section (14). The annular channel (17) is approx. 2 mm wide. It serves to receive the internal thread adapter (20).

According to FIGS. 5 and 6, the internal thread adapter (20) is a tubular body with a central stepped bore (22). The latter comprises a threaded zone (23) and a plurality of bore sections (26, 28, 29). The threaded zone (23) has an internal thread (24), for example a double-threaded right-hand thread with a pitch of 5 mm, cf. DIN 13090, Part 2. The threaded zone (23) is adjoined by a front bore section (26) which has a diameter which is slightly smaller than the core diameter of the internal thread (24).

The bore section (26) is followed by an adapter base (27) with a central bore (28). The adapter base (27) is an inner flange which sits with play in the annular channel (17) of the syringe barrel (10). Below the adapter base (27) there is a rear bore section (29) which ends towards the cylinder base (11), for example in a planar end surface (38).

The internal thread adapter (20) essentially has a cylindrical outer wall (31) on which a plurality of longitudinal webs (32), for example 12 of the same, are arranged, cf. FIGS. 1 and 6. The rectilinear longitudinal webs (32) are distributed equidistantly over the circumference of the outer wall (31). They begin approx. 0.8 mm below the upper end surface (21) with a 45° bevel and reach as far as the lower end surface (38). The upper end surface (21) is rounded at its outer edge, for example with a radius of 0.5 mm.

The individual longitudinal webs (32) have a semicircular cross section, wherein the chord length thereof, positioned on the base of the cross section, is approx. half the length of the arc length of the respective gap (33) between two adjacent longitudinal webs (32).

An elastic rubber stopper (40) sits on the Luer outer cone (12), cf. FIG. 5, said rubber stopper sealing the outer cone (12) in a tight and sterile manner. The rubber stepper (40), which can also be manufactured from a flexible plastic, is divided into two regions: a sealing region (41) and a gripping region (51). The sealing region (41) comprises a tubular section, the outer and inner walls of which each form at least approximately a frustoconical surface. The inner wall (48) has a cone angle with a cone angle tapering of 1:16⅔, cf. Luer inner cone according to DIN 13090, Part 1. The outer wall (46) has a cone angle of approx. 3 degrees, with the imaginary tip of the cone, for example according to FIG. 5, being located below the sealing region in the region of the syringe barrel (10). The blind hole recess (42) belonging, to the inner wall, cf. also FIG. 4, has a base (43) which is curved spherically into the discharge bore (13). If appropriate, a for example conical pin (44) is integrally formed on the base (43), said pin, bearing tightly in some regions, projecting into the discharge bore (13), cf. the dashed-line illustration in FIG. 5.

The blind hole recess (42) projects with approx. one fifth of its length into the adjacent gripping region (51).

The gripping region (51) is substantially in the shape of a conical bolt, on the outer wall (55) of which a multiplicity of gripping webs (56), for example ten gripping webs, are integrally formed.

The outer wall (55) has a cone angle of approx. 5 degrees. The gripping webs (56) have a constant cross section over their entire length. The cross section is in the shape of a circular section, the height of which, for example, is one third of the corresponding diameter of the circle. The chord length, which is placed in the cross section base of the gripping webs (56), is approx. three times the length of the arc length of the respective gap between two adjacent gripping webs (56).

The gripping webs (56) are spherically rounded downwards and form an axial collar (57). In the region of the cellar (57), the outside diameter tapers to the outer wall (46) of the sealing region (41) by approx. 66% of the maximum diameter of the gripping region (51). The gripping region (51) has an upper end surface (52) which is labelled, for example and inter alia according to FIG. 7. The end surface (52) is adjoined towards the gripping webs (56) by a circular edge (53) which protrudes slightly beyond the gripping webs (56).

After being placed onto the outer cone (12), the rubber stopper (40) projects with the sealing region (41) fully into the threaded zone (23) of the internal thread adapter (20), but without touching the base of the threaded zone (23). In the process, the rubber stopper (40) is expanded radially to such an extent that it partially touches individual turns of the thread (24), cf. FIG. 8. The rubber stopper (40) bears with an increased tension force in the front region of the outer cone end, since the wall thickness of the rubber stopper is greater in the gripping region (51) than in the sealing region (41).

The base (43) makes contact here in the axial direction with the upper end surface (18) of the outer cone (12) in a, for example, planar annular surface. In addition, the lower gripping web ends and, if appropriate, also the collar (57) rest on the upper end surface (21) of the internal thread adapter (20).

According to FIG. 2, the safety cap (60) is pulled over the fitted combination, illustrated in FIG. 1, comprising the syringe barrel (10), the internal thread adapter (20) and the rubber stopper (40). The initially single-part safety cap (60) comprises a front retaining part (61) and a rear break-off part (81). A break-off zone (97) is located between the two parts (61) and (81).

The safety cap (60), cf. FIG. 6, is a cup-shaped body with a base (91), in which the outer walls (63, 83) of the retaining part (61) and of the break-off part (81), which outer walls are located one behind the other, have a frustoconical basic shape. The cone angle of the basic shape is, for example, one degree. The diameter of the outer walls (63, 83) increases at increasing distance from the base (91). On the basic shape there are arranged, for example, twelve retaining webs (64, 84) distributed over the circumference. All of the retaining webs (64, 84), which are aligned, for example, in the longitudinal direction of the safety cap (60), have trapezoidal cross sections. Said partially different cross sections likewise taper towards the base (91).

The retaining part (61) has a frustoconical inner wall (68) the cone angle of which is, for example, 1.2 degrees. The inside diameter of the inner wall (68) increases here towards the break-off zone (97). In the region of the lower end surface (78) of the retaining part (61), the inner wall (68) has a partially encircling latching web (71), cf. also FIGS. 10 and 11. The radially inwardly protruding latching web (71) has a push-on flank (72) and a retaining flank (73). The push-on flank (72) has a cone angle of 40 degrees. The imaginary tip of the cone lies in the region of the break-off zone (97). The retaining flank (73) has a cone angle of 120 degrees. The imaginary tip of said cone lies in the region of the lower end surface (78).

In the lower region, the outer wall (63) of the retaining part (61) has a step (65) of smaller wall thickness. In the longitudinal direction, the step (65) is of a length which corresponds to approx. 33% of the outside diameter there. Said outside diameter is approx. 6% smaller than the diameter of the adjacent outer wall (63). By means of the reduction in wall thickness, by approx. 46%, the retaining part (61) has a greater degree of elasticity in the region of the latching web (71) than in the rest of the retaining part (61). The step (65) which tapers towards the lower end surface (73) has a cone angle of two degrees.

The retaining part (61) is of a length which is greater than the average diameter of the retaining part (61) by the factor of approx. 1.13.

According to FIGS. 10 and 11, the inner wall (68) has two inner webs (75, 86) which extend, for example, over the entire length of the safety cap (60). Said inner webs are interrupted in the break-off zone (97) and in the break-off part (81) by apertures (85) there, cf. FIG. 7. The inner webs (75, 86) begin at the lower end surface (78). They are bevelled in a roof-shaped manner there in the circumferential direction, cf. also FIG. 5. The inner webs (75, 86) each have a trapezoidal cross section, cf. FIGS. 7 and 8. The web width is approximately half the width of the gap (33) between the longitudinal webs (32) of the internal thread adapter (20). The mutually opposite inner webs (75) are at a clear distance which is always slightly larger than the outside diameter of the outer wall (31) of the internal thread adapter (20).

The break-off part (81) has a frustoconical inner wall (88) which has a cone angle of two degrees. The inside diameter of said inner wall (88) tapers towards the base (91). In the outer wall (83), four at least approximately rectangular apertures (85) are located between the retaining webs (64), cf. FIGS. 2, 9 and 11. Said apertures are each located in pairs next to each other while the pairs lie opposite each other. The length of the apertures (85) is at least 60% of the length of the break-off part (81). According to FIG. 2, the apertures (85) are located by more than 50% in front of the rubber stopper (40). In the exemplary embodiment, the apertures (85) together have an area of 52 mm².

The base (91) likewise has two apertures (92). Each aperture is in the shape of a part of a circular ring. They lie mirror-symmetrically with respect to a plane positioned on the centre line (5). Their two opening cross sections together have an area of 4.9 mm².

A conical retaining pin (93) which is stiffened laterally by two mutually opposite triangular supporting webs (95) is located between the two apertures (92). The supposing webs (95) are supported between the apertures (92) on the base (91). They do not reach as far as the lower free end of the retaining pin (93). The retaining pin (93) is of a length which corresponds, for example, to 64% of the average inside diameter of the break-off part (81). Its free end surface (94), with which it rests, when fitted, on the rubber stopper (40), has an area of at least 3.6 mm².

The break-off part (81) is of a length which is larger than the average diameter of the break-off part (81) by the factor of approx. 1.2. Said length corresponds to the average thickness of an index finger.

At least three break-off webs (98) are located between the lower end surface (96) of the break-off part (81) and the upper end surface (62) of the retaining part (61). There are four of them in the present exemplary embodiment. Each break-off web (98) is of the shape of an inclined truncated pyramid, with the base surface of the truncated pyramid lying on the lower end surface (96) of the break-off part (81), cf. FIGS. 5, 6, 2, 10 and 11. The surface of the roof of the truncated pyramid—i.e. the transition surface to the retaining part (61)—is approx. one quarter of the base surface of the truncated pyramid.

Next to in each case two mutually opposite break-off webs (98), cf. FIG. 2, there are two supporting pins (99), which are integrally formed on the break-off part (81), of the size of the break-off webs (98). However, said supporting pins (99) do not touch the upper end surface (62) of the retaining part (61). In the unloaded state, they are at a distance of approx. 0.3 mm from the retaining part (61).

In order to produce the tamperproof seal, the internal thread adapter (20) is first of all pushed onto the outer cone (12) of the syringe barrel (10) in such a manner that its adapter base (27) latches into the annular channel (17). The internal thread adapter (20) now sits on the syringe barrel (10) in a manner such that it is freely rotatable about the centre line (5). The rubber stopper (40) is pushed onto the outer cone (12) until its blind hole base (43) rests on the end surface (18) of the outer cone (12). In a final step, the safety cap (60) is placed over the rubber stopper (40) and the internal thread adapter (20). In the process, the two inner webs (75) of the safety cap (60) enter the corresponding gaps (33) of the internal thread adapter (20). At the end of the pulling-over movement, the retaining pin (93) is positioned centrally on the rubber stopper (40) and, virtually at the same time, the latching web (71) latches with its retaining flank (73) behind the lower end side (78) of the internal thread adapter (20). The safety cap (60) now sits captively and non-releasably on the syringe barrel (10). The tamperproof seal is produced.

The break-off zone (97) is now located approx. 1 mm below the upper end surface (21) of the internal thread adapter (20) and approx. 2.7 mm below the upper end side (18) of the outer cone (12), cf. FIG. 5.

In order to open the syringe barrel (10), first of all the break-off part (81) of the safety cap (60) has to be torn off for releasing purposes. For this purpose, the break-off part (81) is held in the manner of pincers between the thumb and the index finger and is loaded towards the side, cf. FIG. 3. If the bending torque exceeds, for example, 25 Ncm, the break-off webs (98) positioned in the pulling zone, to which the load is applied start to tear off first. As soon as one break-off web (98) has been torn off, the remaining webs also follow.

Since the rubber stopper (40) does not bear against the break-off part (81), it is not released from the outer cone (12), not even if it is temporarily deflected by an angle of up to 20 degrees during the breaking off of the break-off part (81). The rubber stopper (40) is only pulled off if the MRT cartridge is to be used. Until then, it remains sealed in a sterile manner.

In order to pull off the rubber stopper (40), the latter can also be pressed elastically to the side. In the process, it is supported on one side by its collar (57) and/or the lower ends of the gripping webs (56) on the upper end surface (21) of the internal thread adapter (20), which makes it considerably easier to pull it off.

After the rubber stopper (40) has been pulled off, the assembly comprising the retaining part (61) and the internal thread adapter (20) can rotate freely on the syringe barrel (10), which makes it possible to attach infusion instruments, for example, without rotating the syringe barrel (10) itself.

The syringe barrel (10), the internal thread adapter (20) and the safety cap (60) are manufactured for example from plastic. In this case, each component (10, 20, 60) can be produced from a specific material.

In the above text, the terms “at the top” and “at the bottom” are used repeatedly. They relate to the orientation of the component as illustrated in each of FIGS. 1 to 6. What is at the top in said drawings is also at the top of the component.

### LIST OF REFERENCE NUMBERS

5 Centre line
10 Syringe barrel
11 Barrel base
12 Outer cone, Luer outer cone, conical adapter part
13 Discharge bore
14 Truncated cone section
17 Annular channel, conical adapter part

18 End surface, at the top
20 Internal thread adapter, conical adapter part
21 End surface, at the top, at the front
22 Stepped bore
23 Threaded zone
24 Internal thread
26 Bore section, at the front
27 Adapter base
28 Bore
29 Bore section, at the rear
31 Outer wall
32 Longitudinal webs
33 Gaps
38 End surface, at the rear, at the bottom
40 Rubber stopper
41 Sealing region
42 Blind hole recess, conical
43 Blind hole base, base
44 Pin
46 Outer wall
48 Inner wall
51 Gripping region
52 End surface, at the top
53 Edge, radial, at the top
55 Outer wall
56 Gripping webs
57 Collar, axial
60 Safety cap
61 Retaining part
62 End surface, at the top
63 Outer wall
64 Retaining webs, on the outside; structure
65 Step
68 Inner wall
71 Latching web
72 Push-on flank
73 Retaining flank
75 Inner web
76 Bevel, double-sided, roof-shaped
78 End surfaces, at the bottom
81 Break-off part
82 End surface, at the top
83 Outer wail
84 Retaining webs, on the outside
85 Apertures
86 Inner web
88 Inner wall
89 Interior
91 Base
92 Apertures
93 Retaining pin
94 End surface, at the free end of (93)
95 Supporting webs
96 End surfaces, at the bottom
97 Break-off zone
98 Break-off webs
99 Supporting pin

1. Tamperproof seal for a container or a syringe, with a lockable conical adapter which is sealed in a sterile manner by means of a rubber stopper (40) and is protected by a safety cap (60), wherein the safety cap (60), which is pushed non-releasably onto the conical adapter lock, comprises a retaining part (61), a break-off part (81) and a break-off zone, (97) located between said parts (61) and (81),
characterized
in that the break-off part (81) surrounds the rubber stopper (40) without radial contact,
in that the break-off part (81) has a retaining pin (93), the free end of which rests axially on the rubber stopper (40)—when the tamperproof seal is fitted,
in that the break-off part (81) has at least two open apertures (85, 92), and
in that the length of the break-off part (81) corresponds to at least twice the length of the internal thread (24) of the conical adapter lock.

2. Tamperproof seal according to claim 1, characterized in that the lockable conical adapter comprises an outer cone (12) and an internal thread adapter (20) which can be fitted behind the outer cone (12).

3. Tamperproof seal according to claims 1 and 2, characterized in that the retaining pin (93) has a diameter which is smaller than the inside diameter of the discharge bore (13) of the outer cone (12).

4. Tamperproof seal according to claim 1, characterized in that the retaining pin (93) is of a length which corresponds to at least 0.4 times the length of the break-off part (81).

5. Tamperproof seal according to claim 3, characterized in that the retaining pin (93) is in the shape of a truncated cone and is flanked by two mutually opposite supporting webs (95).

6. Tamperproof seal according to claim 1, characterized in that the rear region of the interior (89) of the break-off part (81), in which the retaining pin (93) is located, takes up at least 40% of the entire interior volume of the break-off part (81).

7. Tamperproof seal according to claim 1, characterized in that the break-off part (81) has in each case at least one aperture (85, 92) in its radial outer wall (83) and on its base (91), with the sum of the cross sections of all of the apertures (85, 92) corresponding to at least ten times the bore cross section of the discharge bore (13).

8. Tamperproof seal according to claim 1, characterized in that the outer wall (63) of the retaining part (61) has a structure (64) at least in some regions.

9. Tamperproof seal according to claim 1, characterized in that the apertures (85) of the radial outer wall (13) extend in the longitudinal direction and are of a length which corresponds to at least 60% of the length of the break-off part (81).

10. Tamperproof seal according to claim 1, characterized in that the internal thread adapter (20) is arranged in a form-fitting manner at the rear end of the outer cone (12) in the longitudinal direction of the conical adapter.

11. Tamperproof seal according to claim 1, characterized in that there is an encircling annular channel (17) for the form-fitting fastening of the internal thread adapter (20) to the rear end of the outer cone (12).

12. Tamperproof seal according to claim 1, characterized in that four break-off webs (98) are arranged in the break-off zone (97) between the retaining part (61) and the break-off part (81).

13. Tamperproof seal according to claim 12, characterized in that each break-off web (98) is of the shape of an inclined truncated pyramid, with the base surface of the truncated pyramid lying on the break-off part (81).

14. Tamperproof seal according to claim 12, characterized in that the break-off webs (98) on the retaining part (61) tear off if the bending torque acting on the break-off part (81) is greater than 25±10 Ncm.
